(19) United States
(12) Patent Application Publication
(10) Pub. No.: US 2022/0266060 A1
(43) Pub. Date: Aug. 25, 2022

(54) METHODS AND APPARATUSES FOR FIDUCIAL REGISTRATION OF INTRAORAL POSITIONING DEVICES FOR RADIATION THERAPY OF HEAD AND NECK CANCER

(71) Applicant: Radtec Medical Devices, Inc., San Carlos, CA (US)

(72) Inventors: Joseph W. Maresca, Jr., Sunnyvale (US); Ross Holman, Menlo Park, CA (US); Brian Knott, Palo Alto, CA (US)

(21) Appl. No.: 17/629,719

(22) PCT Filed: Jul. 23, 2020
(86) PCT No.: PCT/US20/43316

§ 371 Date: Jan. 24, 2022

Related U.S. Application Data
(60) Provisional application No. 62/878,112, filed on Jul. 24, 2019.

(51) Int. Cl. A61N 5/10; B33Y 80/00

(52) U.S. Cl. CPC A61N 5/1049; A61N 5/103; A61N 5/1067; B33Y 80/00

(57) ABSTRACT

A method and apparatuses are disclosed for registration of the patient's head and oral cavity for radiation therapy treatment of head and neck cancer. The method and apparatuses comprise an intraoral positioning device (IPD) positioned in a patients mouth. The IPD incorporates fiducials embedded in or on an IPD. The fiducials function as markers for tracking and radiation beam direction. These fiducials may be incorporated manually, using 3D printers, or a combination of both. The fiducials allow better registration of the radiation therapy during treatment and from treatment to treatment, and also allows adaptive adjustment during radiation therapy to minimize the effects of patient movement.

### REFERENCE TO RELATED APPLICATIONS

This application claims priority to U.S. provisional application No. 62/878,112, filed on Jul. 24, 2019 entitled “Fiducial Registration of Intraoral Positioning Devices For Radiation Therapy of Head and Neck Cancer,” which is incorporated by reference herein.

### FIELD OF THE INVENTION

The present invention relates to methods and apparatuses for inclusion and use of fiducials in intraoral positioning devices for registration of radiation therapy of head and neck cancer.

### BACKGROUND OF THE INVENTION

Over the last 10 years, head and neck cancer diagnoses have increased annually by ten to fifteen percent. The rise in occurrence is largely the result of the increase in HPV infections. Radiation therapy (e.g. Intensity modulated radiation therapy treatment, IMRT, or intensity modulated proton therapy, IMPT) is a primary treatment modality along with surgery and chemotherapy for such cancers. In IMRT and IMPT, patient target locations are treated with small beams of high intensity radiation that are delivered from multiple directions to conform to the shape of the tumor while avoiding healthy anatomic structures to reduce radiation exposure.

In order for radiation therapy to be effective under any radiation therapy treatment modality , i.e., to reduce or remove malignant tumors, the patient should remain motionless and in the same position relative to the radiation during each treatment session and between treatment sessions over the course of many treatment sessions (e.g. 30). Prior devices for radiation treatments, however, have been less than effective for such purposes when it comes to positioning the dental structures (e.g., lower jaw) including the tongue. This is due to the inadequacy of prior methods and products used to make such devices.

It would be advantageous to provide improvements in the method and apparatuses for radiation treatments.

### SUMMARY OF THE INVENTION

Methods and apparatuses (or systems) are disclosed for registration of a patient's head and oral cavity for radiation therapy treatment of the patient's head and neck cancer. In the method and apparatuses, markers or fiducials are secured to or embedded in or on a custom or non-custom intraoral positioning device (IPD). A 3D printer may be used to produce a custom IPD. The markers or fiducials may be applied manually, using 3D printers, or a combination of both. The IPD with embedded fiducials improves registration of the radiation therapy during each treatment and from treatment to treatment. In addition, the IPD with embedded fiducials also enables adaptive adjustment during radiation therapy to minimize the effects of patient movement. Further, the use of fiducials allows reliable automation of the registration. Finally, the use of fiducials allows for better planning and targeting of the radiation therapy.

Methods and apparatuses are disclosed for accurately and reliably determining the position of an IPD in a patient's mouth before, during, and after radiation therapy for head and neck cancer using the fiducials (makers), either embedded in or on the IPD. Various embodiments of IPDs incorporating fiducials are disclosed. Further, methods and apparatuses to manufacture IPDs with the fiducials are also disclosed. The fiducials are used to register, either manually or automatically, the radiation beam in real time before and during the radiation treatment.

Methods and apparatuses are disclosed that provide an IPD with fiducials for registration, whereby an upper bite plate and a lower plate of the IPD is positioned in the patient's mouth to enable accurate and reliable delivery of radiation therapy. Additional fiducials may be placed in a tongue device (tongue paddle) of the IPD.

In accordance with an embodiment of the disclosure, a custom IPD is manufactured using 3D printers (or other method of manufacture known to those skilled in the art) and configured to fit within a patient mouth. The IPD is constructed with three or more small carbon-based spheres (or other materials and shapes known to those skilled in the art) embedded in both the lower and the upper bite plates of the IPD. The spheres function as fiducials that are detectable in medical images used to register the radiation beam so that the position of both the lower and the upper bites of a patient can be uniquely determined in three dimensions with respect to the radiation beam, and the top and bottom bites can be uniquely determined in three dimensions with respect to each other. Further, the precise position of the IPD may be accurately determined in two examples of fiducial placement. In the first example, two fiducials in the upper or lower bite sections or plates of the IPD are configured so that those fiducials are not aligned with the direction of the radiation beams. In the second example, three fiducials within the IPD are employed and configured so that the fiducials do not lie in a same plane. In accordance with another embodiment of this disclosure, fiducials may also be used with a non-custom IPD.

In accordance with an embodiment of this disclosure, a method is disclosed for determining the direction of a radiation beam during treatment of a head and/or neck cancer comprising the steps of: introducing an intraoral positioning device into an oral cavity of a mouth of a patient for radiation treatment, wherein the intraoral positioning device includes one or more fiducials configured to function as markers for obtaining radiation coverage with respect to the oral cavity during radiation treatment, thereby targeting a location of head and/or neck cancer relative to the one or more fiducials for the radiation beam; and determining location coordinates of said fiducials relative to the direction coordinates of the radiation beam. The method further comprises aligning the radiation beam with the coordinates of the fiducials during the treatment of head and/or neck cancer. The method further comprises planning a position of the radiation beam for the treatment plan of the patient using the fiducials to obtain a specified coverage area of said cancer.

In accordance with an embodiment of this disclosure, an apparatus is disclosed for radiation therapy for treatment of a head and/or neck cancer of a patient, the apparatus comprising: an intraoral position device configured to be positioned in a mouth of the patient for the radiation therapy; and one or more fiducials positioned in and/or on the intraoral positioning device, wherein the fiducials are configured to function as markers for tracking motion of the intraoral positioning device and/or determining a direction of a radiation beam relative to the one or more fiducials.

In accordance with an embodiment of this disclosure, a method of making an intraoral positioning device for radiation therapy of head and neck cancer, wherein the intraoral positioning device includes one or more markers to determine the position of the device within a patient's mouth, the method comprising: positioning the one or more markers in the intraoral positioning device, wherein the one or more markers includes one or more fiducials that can be identified in images for the radiation therapy. The IPD may be a custom or non-custom IPD.

### BRIEF DESCRIPTION OF THE DRAWINGS

FIGS. 1(a)-(c) illustrate plan, front and side views of an example custom IPD with a vertical tongue depressing paddle for a tongue down position.

FIGS. 2(a)-(c) illustrate plan, front and side views of another example custom IPD with a lateral tongue deviation paddle and a full arch.

FIGS. 3(a)-(c) illustrate plan, front and side views of an example non-custom IPD in a medium size mouth open tongue forward (MOTF) configuration for a medium sized oral cavity, or mouth.

FIGS. 4(a)-(c) illustrate plan, front and side views of another example non-custom IPD in a large mouth open tongue down (MOTD), depressed) configuration for a large sized oral cavity.

FIGS. 5(a)-(c) illustrates a plan, rear perspective and front views of another non-custom lateral tongue localizing (LTL) for an average sized oral cavity.

FIGS. 6(a)-(c) illustrate views of various small, medium, and large non-custom IPDs.

FIGS. 7(a)-(c) illustrate the plan and front views of the custom IPD in FIG. 1 with voids or channels positioned inside the solid material of the IPD through the surface that are generated during the manufacturing of the IPD for later insertion of fiducials.

FIGS. 8(a)-(c) illustrate the plan, side and front views of the custom IPD in FIG. 2 with voids positioned inside the solid material of the IPD (that are connected to the surface that are generated during the manufacturing of the IPD for later insertion of fiducials).

FIG. 9 illustrates a sample method of deploying an IPD and delivery radiation treatment to a patient.

### DETAILED DESCRIPTION OF THE INVENTION

As known to those skilled in the art, an intraoral positioning device (IPD) is positioned within a patient's mouth during radiation therapy. The IPD may be a custom or non-custom IPD. A custom IPD allows the most precise radiation treatment of the cancerous tumor/region in a patient or user and minimizes radiation exposure to healthy tissue. The IPDs described herein incorporate fiducials that function as markers for improved radiation treatment (i.e., better registration) including, for example, tracking the motion of the IPD and radiation beam direction. The fiducials are typically permanent but may be removable as known to those skilled in the art.

Better registration of an IPD with respect to the radiation beam has significant advantages for improved radiation therapy of cancer in the head and neck of a patient. The use of fiducials in the IPD provides more accurate targeting of the cancerous tissues for planning of a treatment (e.g., tracking), during execution of each treatment (e.g., radiation beams), and from treatment to treatment. This is because registration and alignment for tracking and/or radiation beam direction is improved as a result of the incorporation of the fiducials. The use of fiducials for improved registration is particularly important because multiple radiation treatments are required and each treatment requires registration of the patient in the same position relative to the radiation beam so that only the targeted cancerous region is treated, i.e., irradiated. This is also important during a treatment if the treatment must be temporarily stopped and re-started.

The use of fiducials allows for real-time adjustment of the radiation beam to account for patient movement during treatment. Adaptively adjusting the radiation beam delivery over time to account for patient movement during treatment will better target the cancerous region during the treatment and will minimize radiation of healthy tissues, and therefore, the side effects resulting from radiation exposure. Even if real-time adaptive radiation delivery is not done, detection of too large a change in the position of the fiducials during a treatment can be easily determined and if needed, radiation can be temporarily stopped during a treatment to re-register the patient.

Additionally, the use of fiducials may allow for the use of smaller margins during treatment, because the cancer tissue can be more accurately targeted and any patient movement can be ascertained in real time and adjusted for during treatment. This allows for a radiation treatment plan with smaller margins, where the margins are healthy tissue that are irradiated to be sure that undetected cancer is also treated, but such margins are typically larger than they need to be to account for movement that often occurs during the treatment. The IPD with fiducials minimizes radiation of healthy tissues and minimizes the side effects resulting from radiation.

Finally, the use of fiducials improves the accuracy of radiation treatment and is easier to obtain, check, maintain, and monitor than the use of non-fiducial methods of registration. It is also easier to establish a simple geometric relationship, which means, as stated above, that the patient registration can be done automatically. A point source gold or carbon-based fiducial is easier to detect in an image and to digitize than a certain point on a tooth or the like.

As indicated above, fiducials can be used with both custom and non-custom IPDs. While custom IPDs provide more reliable, accurate, and safest treatment than non-custom IPDs, IPDs with the addition of fiducials increases the reliability, accuracy, and safety of radiation treatment of both types of IPDs.

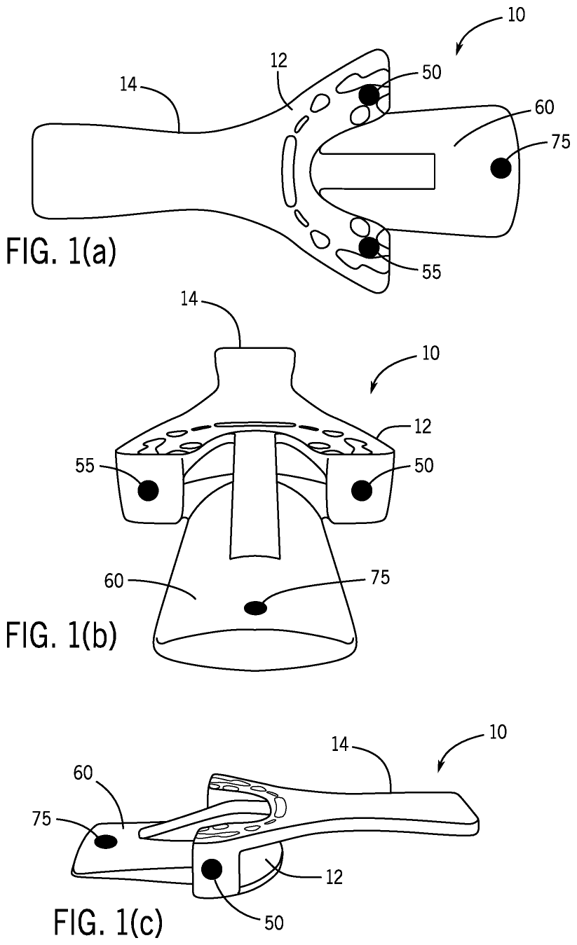

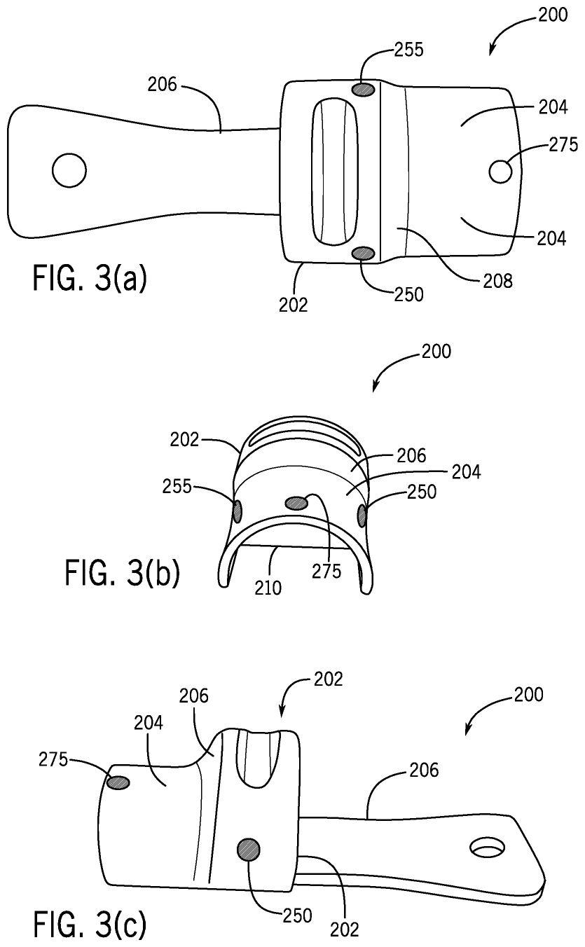

FIGS. 1(a)-(c) and 2(a)-(c) illustrate two example custom IPDs of many possible designs, sizes, and configurations. FIGS. 3(a)-(c)-5(a)-(c) illustrate three example non-custom IPDs of many possible variations. These IPDs (and all disclosed herein) are used to position a patient's (user's) jaw, mouth and soft tissue (e.g., tongue) for radiation planning and/or treatment. Radiation planning and treatment includes intensity-modulated radiation therapy (IMRT), intensity modulate proton therapy (IMPT) or other types of radiation planning and treatment that involves radiation therapy (e.g. CT imaging, X-ray imaging) and non-radiation treatment such as MRI imaging. These IPDs may be one-piece (integral for example) or assembled from pieces as known to those skilled in the art.

FIGS. 1(a)-(c) illustrate a plan, front, and side perspective views, respectively of an example custom IPD 10 as a vertical deviation stent where a patient's or user's tongue is below tongue displacement paddle 60 of IPD 10 in a deployed configuration (in patient). IPD 10 includes engagement member 12, handle 14 and tongue displacement paddle 60. Engagement member 12 and tongue displacement paddle 60 are installed within the cavity of the patient's mouth as known to those skilled in the art. Engagement member 12 is configured as a bite block whereby upper and lower sections of the block are configured to engage the upper and lower arches or dental structures when deployed. Tongue displacement paddle 60 acts as a displacement stent for the tongue, thereby limiting movement of the patient's tongue. (Note IPD may be referred to as a stent herein.) Surface-mounted fiducials 50, 55, and 75 are depicted. Specifically, fiducials 50, 55 are positioned in an opposing configuration and between the upper and lower sections of the engagement member 12 (near the outer surface of the bite block). Fiducial 75 is positioned in the distant portion of tongue displacement paddle 60. In this embodiment, fiducials 50, 55, 75 are embedded inside IPD 10 but they may alternatively be embedded or positioned on the surface of IPD 12 components.

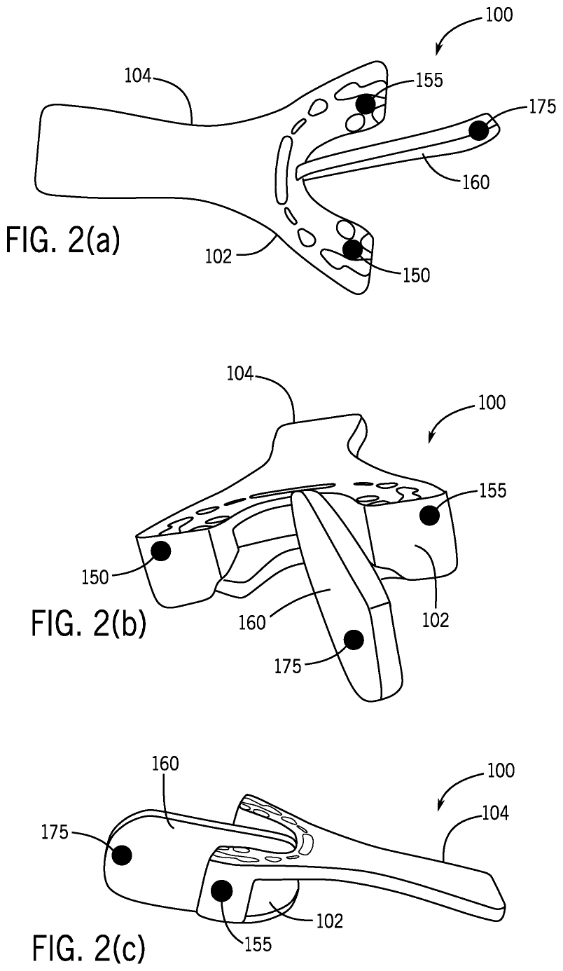

FIGS. 2(a)-(c) illustrates a plan (rear perspective looking forward, back to front), front, and side perspective views, respectively of another custom IPD 100. Similar to the embodiment in FIGS. 1(a)-(c), IPD 100 includes engagement member 102, handle 104 and the tongue displacement paddle 160. Engagement member 102 and tongue displacement paddle 160 are deployed within the cavity of the patient's mouth as known to those skilled in the art. Engagement member 102 is configured as a bite block whereby upper and lower sections of the block are configured to engage the upper and lower arches or dental structures when deployed. Tongue displacement paddle 160 acts as a displacement stent for the tongue, thereby limiting movement of the patient's tongue. In this embodiment, IPD 100 is configured as a lateral stent full arch with a patient's tongue positioned on the left side of the mouth by the tongue displacement paddle 160. Surface-mounted fiducials 150, 155, and 175 are depicted. Fiducials 150, 155 are positioned in an opposing configuration and between the upper and lower sections of the engagement member 102 (near the outer surface of the bite block). Fiducial 175 is positioned in the distant portion of tongue displacement paddle 160. In this embodiment, fiducials 150, 155, and 175 are embedded inside IPD 100 but they may alternatively be embedded or positioned on the surface of IPD 100 components.

FIGS. 3(a)-(c) illustrates a plan, front and side views of example non-custom IPD 200, respectively. IPD 200 is a medium mouth open tongue forward (MOTF) device for a medium sized oral cavity, or mouth. As shown, IPD 200 comprises an engagement member 202 that is configured to engage a patient's anatomical structures (e.g., tissue, tongue and teeth) handle 206. IPD 200 further includes a tong displacement paddle 204 and handle 206 (similar to other example IPDs described herein.) In this example, paddle 204 is a wall that is configured as arc or concave shape. IPD 200 has a transition section 208 between engagement member 202 and tongue paddle 204. Engagement member 202 along with tongue displacement paddle 204 is deployed within the cavity of the patient's mouth as known to those skilled in the art. Engagement member 202, transition section 208 and tongue displacement paddle 204 are configured as arc or concave shape, but having different diameters from one another.

In this example, engagement member 202 has a larger diameter than transition section 208. Engagement section 202 transitions gradually to paddle 204 via transition section 208. Engagement member 202 includes an annular indentation or channel (as a positioning aid) for the maxilla or teeth to engage as described above. The channel is defined by a single wall that is configured as a trough.

IPD 200 also includes a bottom wall 210 that defines the bottom of engagement member 202 whereby its wall and bottom wall 210 define an opening 212 for receiving a patient's tongue. In particular, the patient's tongue is positioned in the opening 270, between the upper and lower incisors, like the user is sticking out their tongue. Surface-mounted fiducials 250, 255, and 275 are shown. Fiducials 250, 255 are positioned in within and along inner segment 202, near the surface of this segment. Fiducial 275 is positioned in the distant portion of segment 204. In this embodiment, fiducials 250, 255, and 275 are embedded inside IPD 200 but they may alternatively be embedded or positioned on the surface of IPD 100 components.

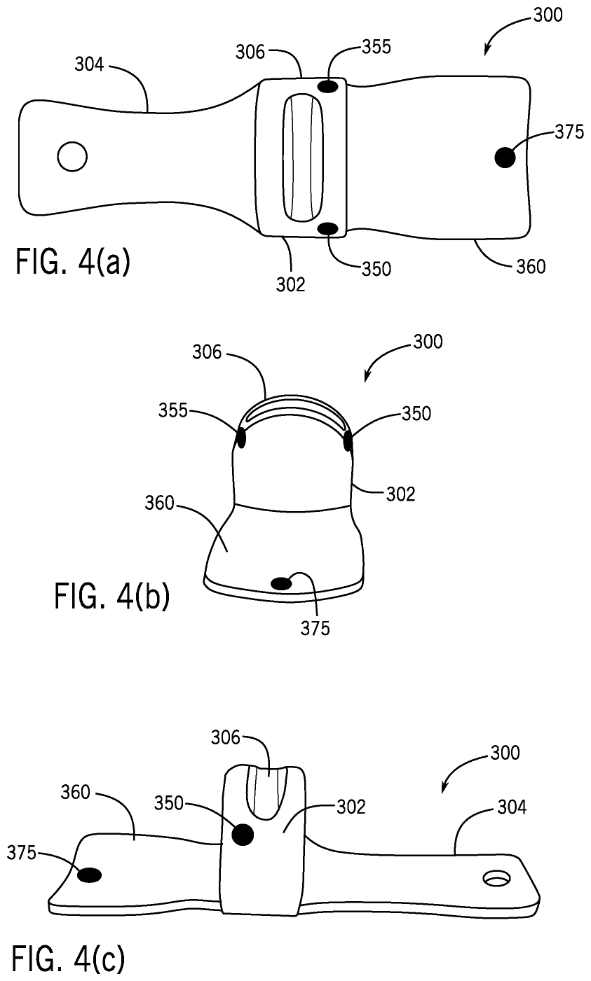

FIGS. 4(a)-(c) illustrates a plan (rear perspective looking forward, back to front), and side views of another example non-custom IPD 300. IPD 300 is a large i.e., a mouth open tongue down MOTD (depressed) stent for a large sized oral cavity. IPD 300 comprises an engagement member 302 that is configured to engage a patient's anatomical structures in his/her mouth including tissue, tongue and teeth as described below, a tongue displacement paddle 360 and handle 304. Handle 304 generally lies the same plane as displacement paddle 360 as shown.

Specifically, engagement member 302 incorporates an annular indentation or channel 306 (as a positioning aid) positioned toward an upper section of engagement member 302 for the maxilla or teeth to engage. The upper section is formed as an arc shape in this example. Channel 306 is defined by an annular shaped wall that is configured as a trough or groove. However, channel 306 may be defined by two, three or more walls that create a groove. A wall may be linear or rounded to a degree. In this example IPD, the patient's tongue is positioned below the tongue displacement paddle 360. Surface-mounted fiducials 350, 355, and 375 are depicted. Fiducials 350, 355 are positioned within the upper section of engagement member 302 toward the front as shown, near the surface of this segment. Fiducial 375 is positioned in the distant portion of tongue displacement paddle 306. In this embodiment, fiducials 350, 355, and 375 are embedded inside IPD 300 but they may alternatively be embedded or positioned on the surface of IPD 300 components.

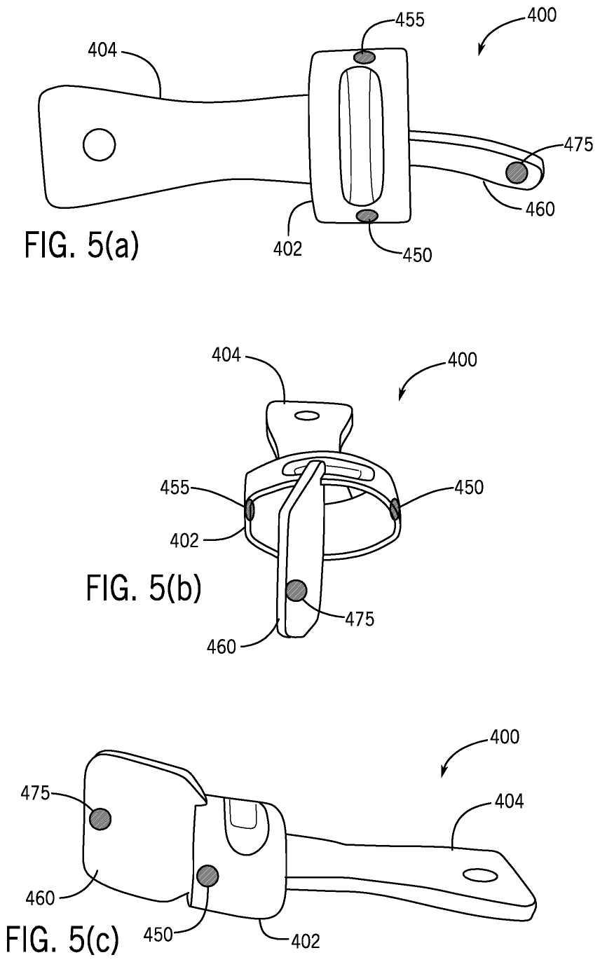

FIGS. 5(a)-(c) illustrates a plan (rear perspective looking forward, back to front), and side views of a non-custom IPD 400. IPD 400 is a lateral tongue localizing (LTL) stent. IPD 400 includes an engagement member 402, handle 404 and tongue displacement paddle 460 as described above. Tongue displacement paddle 460 is configured to position a patient's tongue on the right side of the patient's mouth. Surface-mounted fiducials 450, 455, and 475 are depicted. Fiducials 450, 455 are positioned on the from edges of engagement member 402 toward the front as shown, near the surface. Fiducial 475 is positioned in the distant portion of tongue displacement paddle 460. In this embodiment, fiducials 450, 455, and 475 are embedded inside IPD 400 but they may alternatively be embedded or positioned on the surface of IPD 400 components.

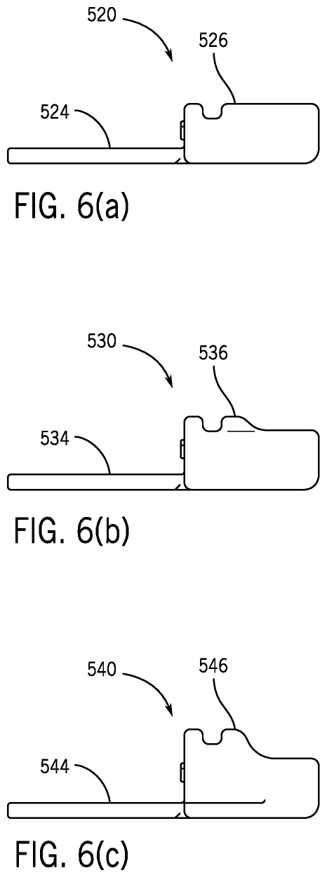

FIGS. 6(a)-(c) illustrate other non-custom IPDs 520, 530, 540, respectively for small, medium and large mouths. The tongue position pieces 526, 536, and 546 are shown. Fiducials may be employed in these IPDs for proper radiation planning and treatment as described herein.

Custom IPDs described herein are set in place by the bite positions of the upper and lower teeth. Once in place in the mouth, a custom IPD will not move due to a direct custom fit within the patient's dental structures, teeth and/or gums. Since the custom IPD is configured to only fit the individual patient, it may be removed between treatments and re-inserted in the same position that it was previously with a high degree of accuracy. By incorporating fiducials into the custom IPD at known locations during the manufacturing process, the positions of the fiducials do not change relative to the oral cavity (and thus tumor location) during and between treatments. The fiducials and custom IPD then become a very accurate and reliable positioning and/or registration aid for radiation therapy planning and treatment (i.e., IPD tracking and/or radiation beam direction). The same is true for non-custom IPDs provided that the non-custom IPD can be positioned in a patient's mouth in the same place from treatment to treatment and held in place during a treatment.

Both the custom and non-custom IPDs have a three-dimensional geometry that will allow fiducials to be positioned in and/or on the IPD itself with vertical and/or horizontal separations and with large enough to provide accurate mathematical estimates of the position of the fiducials relative to the oral cavity and tumor location itself. This is important because more accurate geometric location with respect to the treatment location and radiation beam can be made.

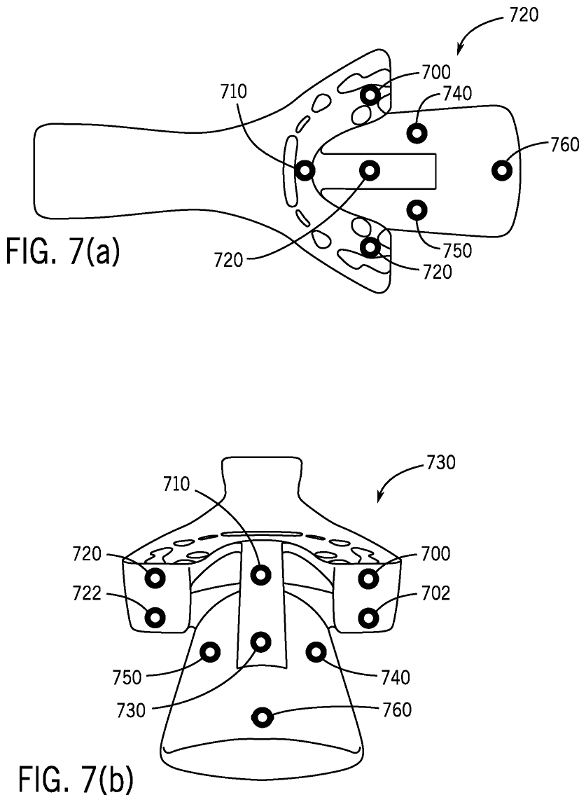

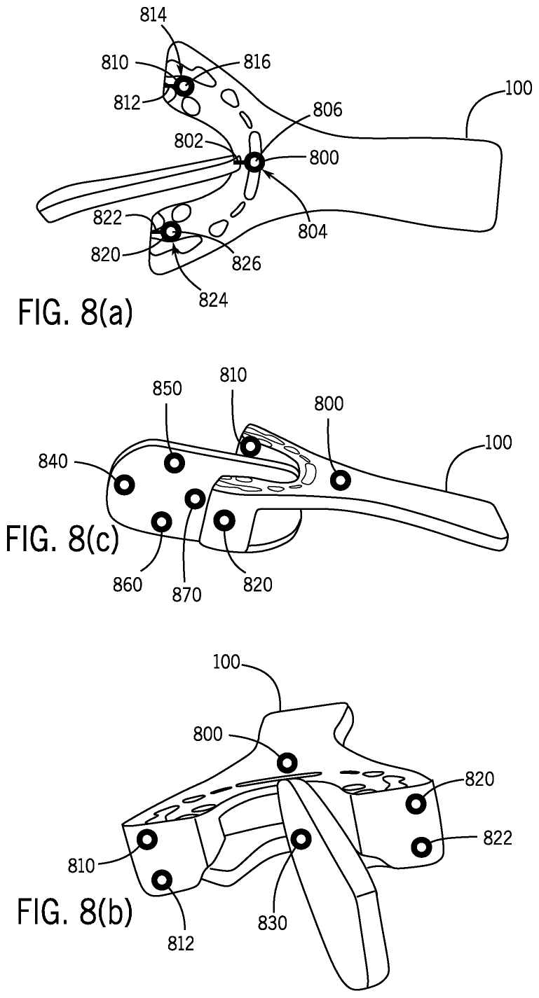

There are many possible locations for three or more fiducials to be positioned in and/or on the IPD itself. This is illustrated in FIGS. 7(a) and 7(b) and 8(a)-(c) using the two custom IPDs shown in FIGS. 1 and 2, respectively.

In FIGS. 7(a) and 7(b), nine fiducials locations are depicted. The fiducial is denoted by the inner circle. The outer circle denotes the initial cavity where the fiducial are placed. Material may be used to fill in the cavity to completely cover the fiducial. The fiducials, particularly the ones on the surface, do not need to be covered if it is made of a safe material cleared or approved for such uses (e.g., gold). The top view in FIG. 7(a) shows only seven of the fiducials. Fiducials 700 and 720 are fiducials posited in a surface cavity as are fiducials 740, 750, 760. Fiducials 710 and 730 are inserted into the solid material. The view in FIG. 7(b) illustrates fiducials 710, 730, 702, 722 inserted into the material and fiducials positioned in a surface cavity 740, 750, 760.

FIGS. 8(a)-(c) depict possible fiducial positions for the oral stent in FIGS. 2(a)-(c). Seven fiducials are illustrated for the IPD in FIG. 8(c). The top view in FIG. 8(a) shows only three of the fiducials. Fiducials 800, 810, and 820 are fiducials positioned in a surface cavity as are fiducials 840, 850, 860, and 870 illustrated in the middle view in FIG. 8(c). The fiducials 812, 830, 822 illustrated in the bottom view in FIG. 8(b) are inserted into the solid material. Different combinations of three or more fiducials will suffice, such as 840, 810, and 820, or 800, 812, and 822, or 800, 812, 820, and 840. The holes 802, 812, 832 leading to the fiducial cavities or channels 804, 814, 824 with the fiducials themselves 806, 816, 826 are depicted. Tubes may be employed as desired for the channels.

There are many methods that can be used to position fiducials in an IPD regardless of whether or not the IPD is being manufactured using a 3D printer. If the IPD is manufactured with a 3D printer, one method is to manufacture the IPD with small voids in the solid regions of the IPD, each of which is connected via an open tube or tunnel to the surface of the IPD. The open tube provides access to the void for the insertion of the fiducial material into the void after completion of the 3D printing. Reference is made to FIGS. 8(a)-(c), where voids and the fiducials are shown. (The voids may each include a tube.) Any spaces remaining in the voids may be filled in with the material used to manufacture the IPD or some other safe, non-toxic material as needed.

Another method is to create small holes near the surface for placement of the fiducials after the manufacturing is completed. Once the fiducials are placed in each location, the hole will be filled in and the fiducial covered with the same material used to manufacture the IPD or some other safe, non-toxic material. A third method is to use a combination of the first two methods. Another method is to use the voids themselves and not connect the voids to the surface with any open tubes. The fiducials can be a solid, a liquid, or a liquid that solidifies once in the void. (Of note, objects of materials may be added to the voids to use as markers.) The fiducials can be a metal or a composite. At a minimum, the fiducials must be small enough to provide accurate reference points to enable radiation planning and treatment as known to those skilled in the art, but large enough to be detectable.

The positions of the fiducials in the IPD relative to the patient's tumor and the direction of the radiation beam must be known. It is helpful to know the position of the fiducials in the IPD during manufacture, but not essential, because registration is best done for each patient to register the IPD relative to the tumor location and anatomical structures. The positions can be determined as part of the manufacturing, after the manufacturing is completed, or when the IPD is inserted into the patients mouth.

A 3D geometric calculation can be made to align the direction of the radiation beam with respect to the IPD using three or more fiducials positioned in the IPD as known to those skilled in the art. If only three fiducials are used, the coordinates of the point of intersection of a plane formed by the three fiducials and the three-dimensional angle of the radiation beam relative to the plane are determined for targeting a certain location of the cancer treatment in the head and neck region. The accuracy of the position estimate improves if more than three fiducials are used. The original purpose of the IPD is to act as a positioning aid and to minimize radiation exposure to healthy tissue. With the addition of fiducials, improved accuracy of radiation delivery is greatly expanded and improved.

The fiducials can be made of any material that functions as a marker as described herein, but must be safe and must be detectable in images used to plan or update the position of the radiation before and/or during the treatment. As discussed above, the fiducials can be inserted into an IPD or onto the surface of an IPD. The material can be an inert metal such as gold, a composite of different density and properties than the IPD, a void, or a void filled with liquid, ink, or dye, or a void in which a solid material is placed. The scattering properties of the materials should be considered in the radiation coverage pattern as known to those skilled in the art. The fiducial can also be a mark inscribed in the IPD or on the surface of an IDP. In general, the fiducials will be a point marker with a unique position. Further, different geometric shapes, sizes and/or patterns can be used for the fiducials.

For accurate positioning of the radiation beam, three or more fiducials should be used and at least one of the fiducials or fiducial locations should be at a different elevation than the other two fiducials. Or said differently, three fiducials should preferably not define a horizontal plane with the tracking sensors and delivery equipment. For custom IPDs, it is desirable to be able to locate each element in the IPD that may move. Thus, fiducials should be placed in the lower and upper bites and in the tongue displacement paddle if it is not rigidly attached to the upper and/or lower arches of the IPD. Different combinations of locations, materials, patterns, and/or positions of the fiducials can be used as well.

The use of fiducials in an IPD provides the capability of accurately and reliably registering a patient's position at the beginning and/or throughout the radiation treatment. Such automation is possible without the use of fiducials. The use of fiducials, which are easy to detect make automatic registration highly feasible. The registration is done by aligning the radiation beam from medical equipment (or systems) mathematically to the position coordinates of the fiducials as known to those skilled in the art. Automatic registration is best done using the custom IPDs because the fiducials in the IPD and the position of the IPD in the patient's mouth does not change from radiation treatment to radiation treatment or during a radiation treatment. Automatic registration can also be done with non-custom IPDs provided that the non-custom IPD is positioned in the mouth relative to natural feature in the patient's mouth or temporary fiducials placed on natural features of the patient's mouth.

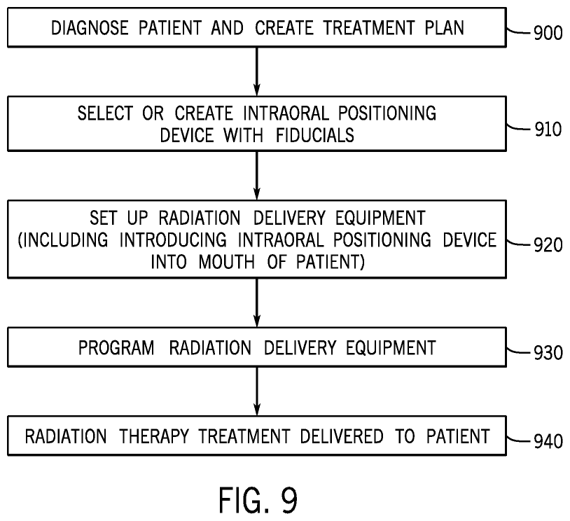

FIG. 9 illustrates a sample method of deploying an IPD and delivery radiation treatment to a patient. Specifically, execution begins at step 900 wherein a patient is diagnosed with head and/or neck cancer and a treatment plan is created to address the cancer, i.e., a tumor. Diagnosis usually includes imaging (e.g., CT, MRI) and the treatment plan typically includes a prescription of an IPD. That is, the treating doctor will prescibe a course of radiation treatment and provide the IPD specification.

Execution proceeds to step 910 wherein an IPD is either selected (non-custom) or and custom IPD is created based on the prescription. The IPD will incorporate fiducials that function as markers for the radiation therapy as described herein.

Execution proceeds to step 920 wherein patient radiation delivery equipment (or parts) is set up. In this respect, the patient is positioned or maneuvered into a treatment position and the IPD with fiducials is introduced into the cavity in the patient's mouth. Any positioning aids are also fitted such as a thermoplastic mask, pillows, etc.

Execution proceeds to step 930 wherein radiation delivery equipment is programmed to deliver radiation therapy to the patient. In this respect, treatment delivery monitoring, control and guidance systems are employed. Fiducial locations are determined by tracking the coordinates of the fiducials. Adjustments may be made to the radiation beam (direction, intensity, etc.) as needed.

Execution proceeds to step 940 wherein radiation therapy treatment is delivered. Real-time adjustments of the radiation beam are made to account for patient movement during treatment such as anatomical changes (along radiation beam path). These adjustments will help improve targeting of the cancerous region during the treatment and will minimize radiation of healthy tissues. Smaller margins during treatment are realized because cancerous tissue can be more accurately targeted.

There are many ways to manufacture or implement fiducials in an IPD. In one example, if the IPD is being manufactured using a 3D printer, fiducials may be positioned on the IPD at the surface or inserted into the IPD itself. If the fiducial is surface mounted, this can be accomplished by creating a small surface cavity during 3D printing process and then placing the fiducial in the cavity after the 3D printing is completed. The same material as the IPD itself may even be used to cover the fiducial as needed. The final step in this process is to smooth out the surface coating.

However, in another example, if a fiducial is inserted inside the IPD, this can be accomplished by (1) creating a small enclosed cavity inside the IPD with a hole or tunnel connecting the cavity to the surface, (2) placing the fiducial in the cavity through the tunnel and (3) filling the tunnel and cavity with the same material that was used to create the IPD. The final step is to smooth out the surface coating at the location of the hole. The tunnel or hole, the cavity, and the fiducial are illustrated in the top view 820 of FIGS. 8(a) (e.g., 812, 814, and 816).

These are only example steps for manufacturing IPDs with fiducials as set forth herein. However, those skilled in the art know that there are many other ways to manufacture or create fiducials within an IPD.

It is to be understood that the disclosure teaches examples of the illustrative embodiments and that many variations of the invention can easily be devised by those skilled in the art after reading this disclosure and that the scope of the present invention is to be determined by the claim(s) below.

What is claimed is:

1. A method for determining the direction of a radiation beam during treatment of a head and/or neck cancer comprising the steps of:
introducing an intraoral positioning device into an oral cavity of a mouth of a patient for radiation treatment, wherein the intraoral positioning device includes one or more fiducials configured to function as markers for obtaining radiation coverage with respect to the oral cavity during radiation treatment, thereby targeting a location of head and/or neck cancer relative to the one or more fiducials for the radiation beam; and
determining location coordinates of said fiducials relative to the direction coordinates of the radiation beam.

2. The method of claim 1 further comprising aligning the radiation beam with the coordinates of the fiducials during the treatment of head and/or neck cancer.

3. The method of claim 1, planning a position of the radiation beam for the treatment plan of the patient using the fiducials to obtain a specified coverage area of said cancer.

4. The method of claim 1 further comprising detecting the fiducials before, during or after the radiation treatment in an image of the intraoral positioning device positioned in the mouth of the patient.

5. The method of claim 1 further comprising detecting the fiducials as a function of time in the images of the intraoral positioning device during radiation treatment.

6. The method of claim 5 wherein the fiducials are detected in real time.

7. The method of claim 6 wherein the detecting of the includes adaptively positioning the radiation beam during said radiation treatment using the fiducials.

8. The method of claim 1 further comprising updating the direction of the radiation beam in real time during the radiation treatment to account for movement of the fiducials of the intraoral positioning device.

9. The method of claim 8 further comprising delivering the radiation treatment with respect to the real-time update of the position of the fiducials.

10. The method of claim 1 further comprising determining three dimensional coordinates of the intraoral positioning device of the radiation beam using the fiducials.

11. The method of claim 1 registering the position of a lower bite plate of the intraoral device relative to the position of an upper bite plate of the intraoral positioning device using the fiducials.

12. The method of claim 1 wherein the one or more fiducials are configured in a pattern from which the position the intraoral positioning device are determined.

13. The method of claim 1 wherein the intraoral positioning device is a custom intraoral positioning device made for the patient.

14. The method of claim 1 wherein the intraoral positioning device is a non-custom intraoral positioning device.

15. The method of claim 14 wherein the non-custom intraoral positioning device are made in a plurality of sizes.

16. An apparatus for radiation therapy for treatment of a head and/or neck cancer of a patient, the apparatus comprising:
an intraoral position device configured to be positioned in a mouth of the patient for the radiation therapy; and
one or more fiducials positioned in and/or on intraoral positioning device, wherein the fiducials are configured to function as markers for tracking motion of the intraoral positioning device and/or determining a direction of a radiation beam relative to the one or more fiducials.

17. The apparatus of claim 16 wherein the one or more fiducials include first, second and third fiducials positioned on said intraoral positioning device.

18. The apparatus of claim 17 wherein the first, second and third fiducials are positioned within the intraoral positioning device so that the first, second and third fiducials do not define a plane that is aligned with the radiation beam.

19. The apparatus of claim 17 wherein the one or more fiducials are positioned in an upper bite plate and in a lower bite plate of the intraoral positioning device.

20. The method of claim 16 wherein the intraoral positioning device is a custom intraoral positioning device made for the patient.

21. The method of claim 16 wherein the intraoral positioning device is a non-custom intraoral positioning device.

22. The method of claim 21 wherein the non-custom intraoral positioning device are made in a plurality of sizes.

23. A method of making an intraoral positioning device for radiation therapy of head and neck cancer, wherein the intraoral positioning device includes one or more markers to determine the position of the device within a patient's mouth, the method comprising:
positioning the one or more markers in the intraoral positioning device, wherein the one or more markers includes one or more fiducials that can be identified in images for the radiation therapy.

24. The method of claim 23 wherein the intraoral positioning device is made using 3D printing methods.

25. The method of claim 23 wherein the one or more markers are one or more voids in the intraoral positioning device.

26. The method of claim 23 wherein the one or more markers are one or more objects of material.

27. The method of claim 26 wherein the one or more objects are materials different than material of the intraoral positioning device.

28. The method of claim 26 wherein the material of the one or more objects comprise a liquid, metal, cloth, ink, dye or gold positioned within or on the intraoral positioning device.

29. The method of claim 25 further comprising adding material to the one or more voids.

30. The method of claim 29 wherein the material may be a liquid, ink, dye, composite, or metal.

31. The method of claim 30 wherein the added material is different than material of the intraoral positioning device.

32. The method of claim 23 further comprising one or more objects added to said one or more voids.

33. The method of claim 32 wherein the material includes a liquid, ink, dye, composite, carbon or metal.
